(19) United States
(12) Patent Application Publication
(10) Pub. No.: US 2018/0318147 A1
(43) Pub. Date: Nov. 8, 2018

(54) METHOD FOR DETECTING INFANT'S URINATION

(71) Applicant: Dongguan Southstar Electronics Limited, Dongguan City (CN)

(72) Inventors: Jun Yi, Dongguan City (CN); Zhihua He, Dongguan City (CN); Fangyi Cai, Dongguan City (CN); Minghui Du, Dongguan City (CN); Weimin Chen, Dongguan City (CN); Zhantu Zheng, Dongguan City (CN); Jianchao Xu, Dongguan City (CN)

(21) Appl. No.: 15/588,683

(22) Filed: May 7, 2017

(51) Int. Cl. A61F 13/42

(52) U.S. Cl. CPC A61F 13/42; A61F 2013/424

(57) ABSTRACT

A method for detecting an infant's urination includes: determining that Condition 1 is satisfied if a relative humidity value collected lastly is greater than humidity values of previous N times and is 1% rh˜3% rh greater than a minimum of the previous N times; determining that Condition 2 is satisfied and the first urination has happened if humidity values collected in continuous M times satisfy Condition 1, and the humidity value collected in the Mth time is 3% rh˜6% rh greater than a minimum of the previous N+M times; starting to detect a second urination when humidity variation becomes steady; and determining that the second urination has happened if a time for humidity to increase 0.1% rh is less than a half of a previous time for humidity to increase 0.1% rh, and a next time for humidity to increase 0.1% rh is also less than a half of the previous time.

### BACKGROUND OF THE INVENTION

### 1. Technical Field

The present invention relates to nursing of infants, and more particularly to a method for detecting an infant's urination.

### 2. Description of Related Art

When having their infants using diaper pants or diapers, parents tend to touch the diaper with hands frequently in order to determine whether the diaper is too wet ant needs to be replaced. Disadvantages related to this practice are many. First, manual test is time consuming and not accurate. Secondly, manual test is impossible to be conducted in a continuous manner, and late knowing about wetness can lead to a baby's diaper rash. Thirdly, frequent testing touches may upset the infant.

One method for indicating wetness of diapers available in the market needs parents' frequent check. This method wastes time and may cause babies' skin allergy or inflammation if parents fail to detect wetness timely. Another approach to this involves placing a test probe on a diaper's stay-dry layer (the inner surface of a diaper). However, the test probe contacting human skin directly may make infants uncomfortable and needs frequent cleaning. Still another existing scheme uses a humidity detector to measure humidity. This method has an alarm triggered when the measured humidity reaches a threshold. However, the known method has only one urination point record, and a good diaper may remain not reaching the threshold after several times of urination, making it difficult to learn the infant's urinary habit, and requiring more time to reach the threshold.

### SUMMARY OF THE INVENTION

In view of this, the objective of the present invention is to address the shortcomings of the prior art by providing a method for detecting an infant's urination, which allows a user to accurately know the infant's urinary habit.

For achieving the foregoing objective, the present invention adopts the following technical scheme:

A method for detecting an infant's urination comprises the following steps:

I. Determining a first urination:

(1) determining that Condition 1 is satisfied if a humidity value collected by a humidity sensor lastly is greater than humidity values of previous N times and is 1% rh˜3% rh greater than a minimum among the humidity values of the previous N times;

(2) determining that Condition 2 is satisfied and the first urination has happened if humidity values collected in continuous M times satisfy Condition 1, and the humidity value collected in the Mth time is 3% rh˜6% rh greater than a minimum among the humidity values of the previous N+M times; and

(3) starting to detect a second urination when variation of the humidity values becomes steady; and

II. Determining the second urination: determining that second urination has happened after the variation of the humidity becomes steady when a time t1 for the relative humidity to increase every 0.1% rh is less than a half of a previous time t0 for the relative humidity to increase every 0.1% rh (i.e. t1<0.5×t0), and a next time t2 for the relative humidity to increase every 0.1% rh is also less than a half of the previous time t0 (t2<0.5×t0).

As a preferred scheme, N and M in Step I are both 7.

As a preferred scheme, a definition of the variation of the humidity values becomes steady in Step (3) is that a time for the relative humidity to increase every 0.1% rh is more than 20 seconds.

As a preferred scheme, an interval for collecting the values of the relative humidity is between 0.5 and 3 seconds.

The present invention has significant advantages and beneficial effects over the prior art. Particularly, it is learned from the foregoing technical scheme that:

the present invention uses a humidity sensor to measure relative humidity, so the infant's urination can be instantly detected, and it can detect at least two times of urination on a single diaper, so as to help the user to learn the infant's urinary habit. In addition, since a good diaper has its surface staying dry after a single time of urination, such a diaper is usually not to be replaced for a single time of urination. The present invention capable of detecting at least two times of urination thus helps to reduce unnecessary diaper waste.

The invention as well as a preferred mode of use, further objectives and advantages thereof will be best understood by reference to the following detailed description of illustrative embodiments when read in conjunction with the accompanying drawings.

### BRIEF DESCRIPTION OF THE DRAWINGS

FIG. 1 shows Condition 1 for determining a first urination according to one preferred embodiment of the present invention;

FIG. 2 shows Condition 2 for determining the first urination according to the preferred embodiment of the present invention;

FIG. 3 is a graph showing increase of humidity during the first urination according to the preferred embodiment of the present invention;

FIG. 4 is a graph showing increase of humidity during a second urination according to the preferred embodiment of the present invention; and

FIG. 5 is a flowchart of the disclosed method according to the preferred embodiment of the present invention.

### DETAILED DESCRIPTION OF THE INVENTION

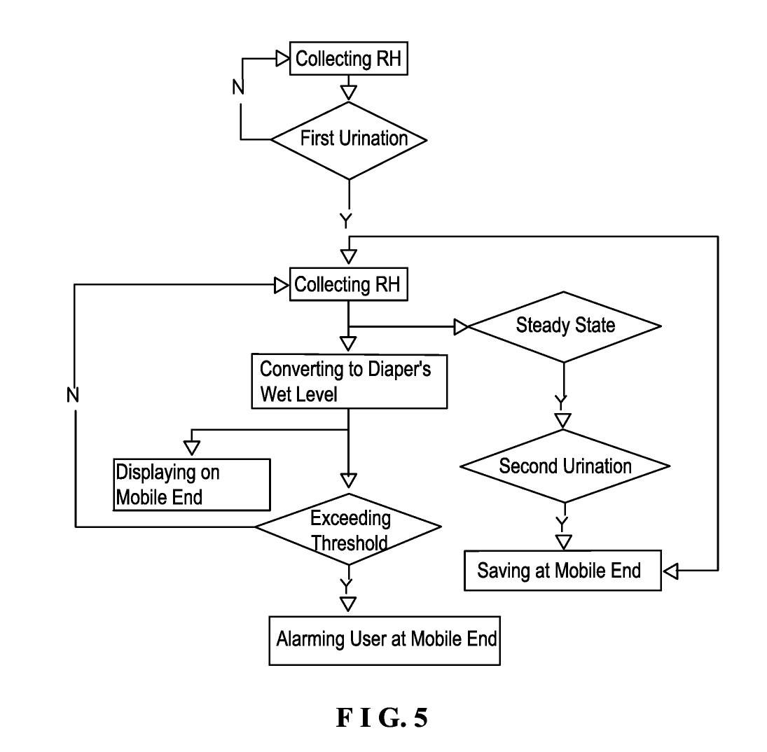

The present invention discloses a method for detecting an infant's urination. As shown in FIG. 5, it comprises the following steps:

I. Determining a first urination:

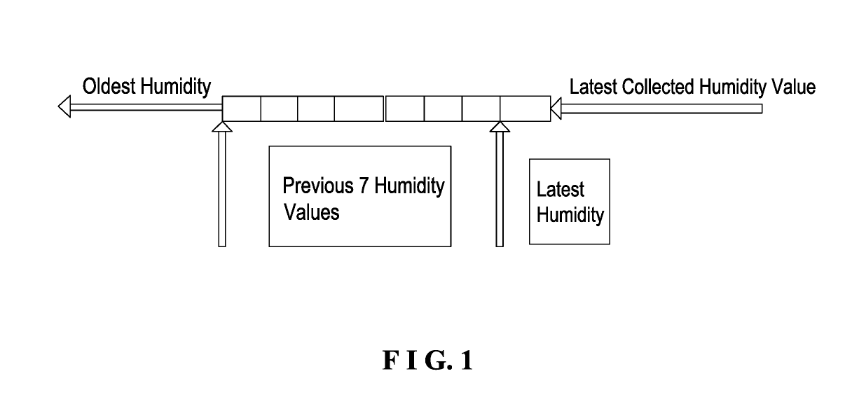

In Step (1), it is determined that Condition 1 is satisfied if a relative humidity value collected by a humidity sensor lastly is greater than humidity values of previous N times and is 1% rh˜3% rh greater than a minimum among the humidity values of the previous N times. In the present embodiment, N is 7. Thus, as shown in FIG. 1, when the humidity sensor collects a new value of relative humidity that is greater than the counterparts of the previous 7 times, and is 1% rh˜3% rh larger than a minimum among the humidity values of the previous 7 times, it is determined that Condition 1 is satisfied.

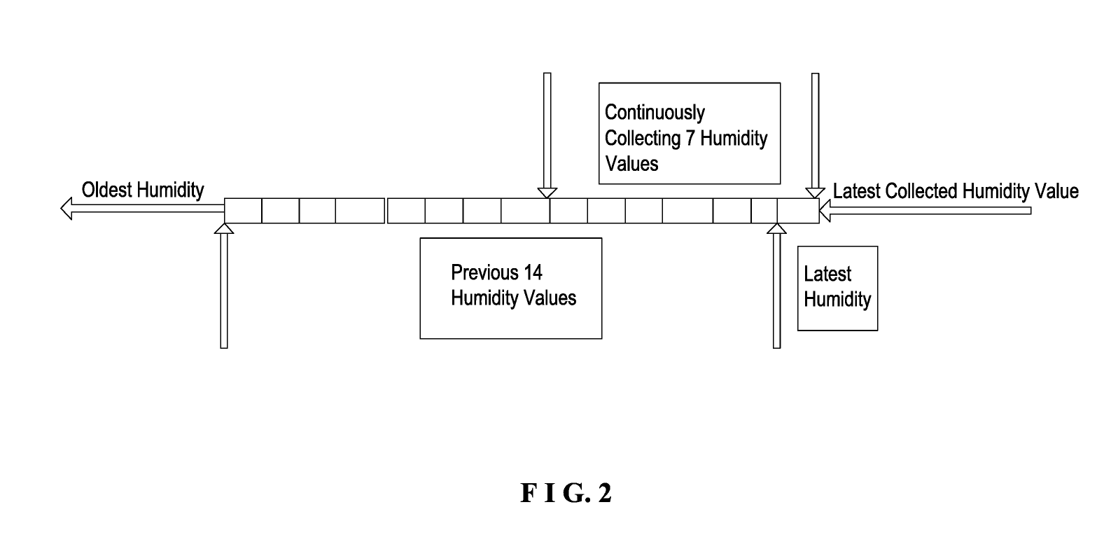

In Step (2), it is determined that that Condition 2 is satisfied and the first urination has happened if humidity values collected in continuous M times satisfy Condition 1, and the humidity value collected in the Mth time is 3% rh˜6% rh greater than a minimum among the humidity values of the previous N+M times. In the present embodiment, M is also 7, so as shown in FIG. 2, when the humidity values collected in continuous 7 times satisfy Condition 1, and the humidity value collected in the 7th time is 3% rh˜6% rh greater than a minimum among the humidity values of the previous 14 times, it is determined that Condition 2 is satisfied and the first urination has happened.

In Step (3), when variation of the humidity values becomes steady, the method starts to detect a second urination. The definition of the variation of the humidity values becomes steady is that a time for the relative humidity to increase every 0.1% is more than 20 seconds. The interval for collecting the values of the relative humidity is between 0.5 and 3 seconds.

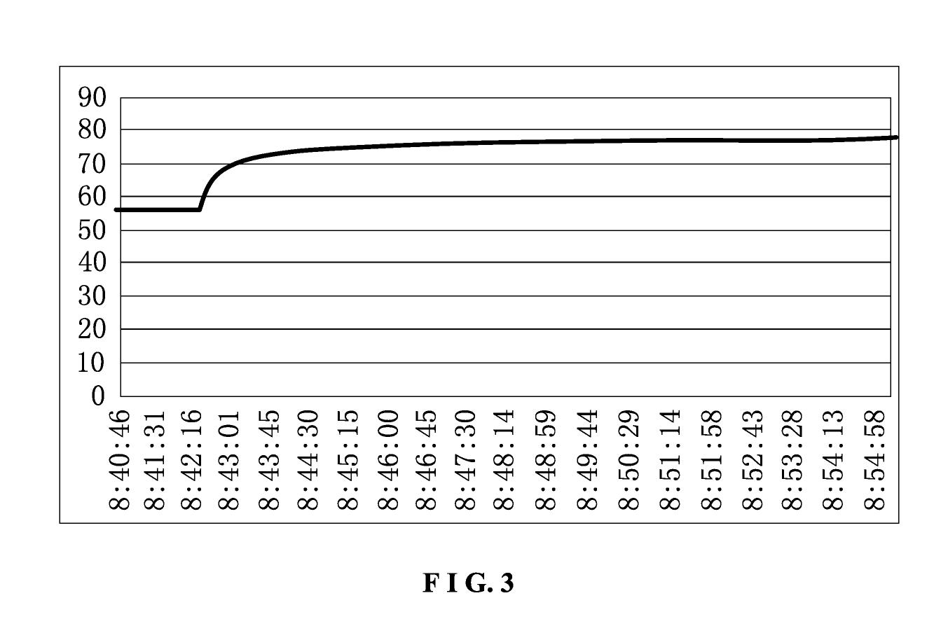

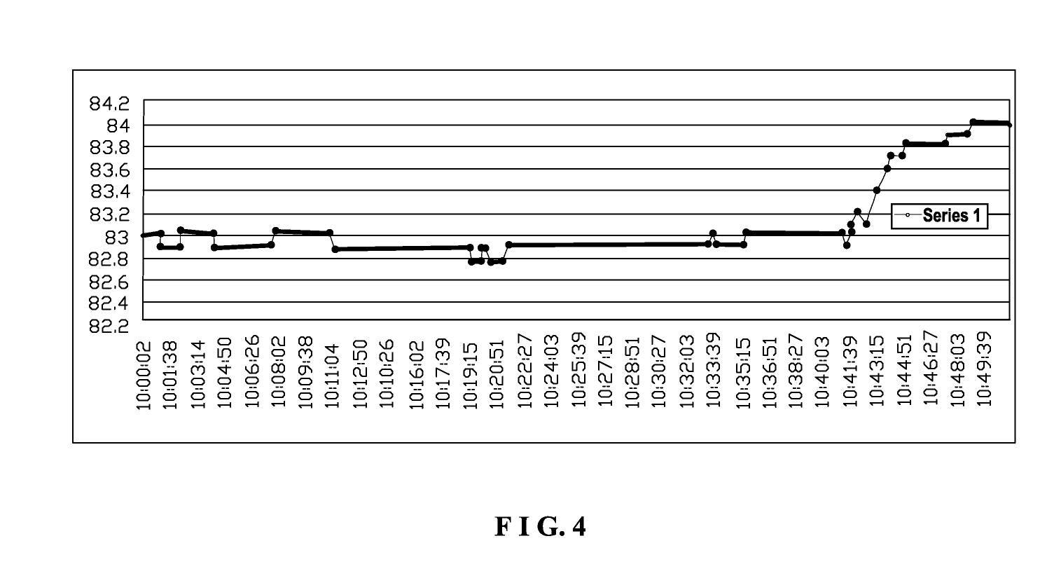

After the first urination, the infant will urinate again, and for the second time of urination, the method for determining the first urination is no more useable. Thus, a separate step is designed to determine the second urination. FIG. 3 shows increase of humidity during the first urination. FIG. 4 shows increase of humidity during the second urination. From FIG. 3, it is learned that the relative humidity increases significantly during the first urination and the aforementioned algorithm is usable. However, from FIG. 4 it is clear that the humidity increases in a breadth of only 0.1% rh, so the step as described below is designed for determining the second urination.

II. Determining the second urination: determining that second urination has happened after the variation of the humidity becomes steady when a time t1 for the relative humidity to increase every 0.1% rh is less than a half of a previous time t0 for the relative humidity to increase every 0.1% rh (i.e. t1<0.5×t0), and a next time t2 for the relative humidity to increase every 0.1% rh is also less than a half of the previous time t0 (t2<0.5×t0).

The foregoing ranges such as 1% rh˜3% rh, and 3% rh˜6% rh, and the interval for collecting humidity (0.5 s˜3 s) may be set differently at a mobile end for different brands of the diapers in use.

The design point of the present invention is that it uses a humidity sensor to measure relative humidity, so the infant's urination can be instantly detected, and it can detect at least two times of urination on a single diaper, so as to help the user to learn the infant's urinary habit. In addition, since a good diaper has its surface staying dry after a single time of urination, such a diaper is usually not to be replaced for a single time of urination. The present invention capable of detecting at least two times of urination thus helps to reduce unnecessary diaper waste.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A method for detecting an infant's urination, comprising the following steps:
I. Determining a first urination:
(1) determining that Condition 1 is satisfied if a relative humidity value collected by a humidity sensor lastly is greater than humidity values of previous N times and is 1% rh˜3% rh greater than a minimum among the humidity values of the previous N times;
(2) determining that Condition 2 is satisfied and the first urination has happened if humidity values collected in continuous M times satisfy Condition 1, and the humidity value collected in the Mth time is 3% rh˜6% rh greater than a minimum among the humidity values of the previous N+M times; and
(3) starting to detect a second urination when variation of the humidity values becomes steady; and
II. Determining the second urination: determining that second urination has happened after the variation of the humidity becomes steady when a time t1 for the relative humidity to increase every 0.1% rh is less than a half of a previous time t0 for the relative humidity to increase every 0.1% rh (i.e. t1<0.5×t0), and a next time t2 for the relative humidity to increase every 0.1% rh is also less than a half of the previous time t0 (t2<0.5×t0).

2. The method of claim 1, wherein values of N and M in Step I are both 7.

3. The method of claim 1, wherein a definition of the variation of the humidity values becomes steady in Step (3) is that a time for the relative humidity to increase every 0.1% rh is more than 20 seconds.

4. The method of claim 1, wherein an interval for collecting the values of the relative humidity is between 0.5 and 3 seconds.
